(19) United States
(12) Patent Application Publication
(10) Pub. No.: US 2024/0238466 A1
(43) Pub. Date: Jul. 18, 2024

(54) ADAPTER AND APPARATUS FOR DISINFECTING DEVICES THAT ARE TO BE OPERATED BY A HUMAN HAND

(71) Applicant: RONOTIC AG, St. Leon-Rot (DE)

(72) Inventor: Cem Koc, Walldorf (DE)

(21) Appl. No.: 17/928,002

(22) PCT Filed: May 28, 2021
(86) PCT No.: PCT/EP2021/000070

§ 371 Date: May 30, 2023

(30) Foreign Application Priority Data
May 28, 2020 (DE) DE10 2020 003 558
May 28, 2020 (DE) DE20 2020 002 592
Jun. 26, 2020 (DE) DE10 2020 004 100
Jun. 26, 2020 (DE) DE20 2020 002 907

(51) Int. Cl. A61L 2/26; A61L 2/10

(52) U.S. Cl. CPC A61L 2/26; A61L 2/10; A61L 2202/14

(57) ABSTRACT

An adapter and an apparatus for disinfecting devices to be operated by the human hand, in particular card readers, which, in the form of a mobile or stationary terminal, are used for the cashless purchase of goods using the electronic cash method, in which a PIN code must be entered upon payment. To enter the PIN code, these EC card readers have a PIN pad with keys or a touchscreen with displayed keys. A housing of the EC card reader is laterally enclosed in the region of the keypad or of the touchscreen by two opposing legs which are interconnected below the housing. At least one portion of one of the legs projects beyond the housing on the keypad side or on the touchscreen side. At least one UV light source irradiates the whole of the keypad or touchscreen is arranged in the at least one portion.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2021/000070, which was filed on May 28, 2021, which application claimed priority from each of the following German applications: DE 10 2020 003 558.2 (filed May 28, 2020), DE 20 2020 002 592.5 (filed May 28, 2020), DE 10 2020 004 100.0 (filed Jun. 26, 2020) and DE 20 2020 002 907.6 (filed Jun. 26, 2020).

### FIELD OF THE INVENTION

A field of the invention concerns devices that accept human entry of personal identification numbers (PIN) via a keypad or touchscreen.

### SUMMARY OF THE INVENTION

A preferred embodiment provides an adapter for EC card readers for the cashless purchase of goods via a manual PIN entry through a keypad or a touchscreen. The adapter is arranged in a housing of the EC card reader. The adapter includes at least two opposing legs that laterally enclose the housing of the EC card reader in the region of the keypad or touchscreen. The legs are interconnected below the housing. At least one portion of at least one of the legs projects beyond the housing on a side of the keypad or the touchscreen. At least one UV light source is arranged in the at least one portion and irradiates an entirety of the keypad or touchscreen.

### BRIEF DESCRIPTION OF THE DRAWINGS

The invention is explained below with respect to the following figures:

FIG. 1 is a perspective view of an EC card reader fitted with the adapter according to the invention;

FIG. 2 is a user perspective view of the FIG. 1 EC card reader fitted with the adapter according to the invention;

FIG. 3 is s side view of the FIG. 1 1 EC card reader fitted with the adapter according to the invention;

FIG. 4 is perspective view of an EC card reader fitted with the adapter according to an embodiment of the invention; and

FIG. 5 is a view of the adapter of FIG. 5.

### DETAILED DESCRIPTION OF THE PREFERRED EMBODIMENTS

The invention relates to an adapter for EC card readers which, in the form of a mobile or stationary terminal, are used for the cashless purchase of goods using the electronic cash method, in which a PIN code must be entered upon payment. To enter the PIN code, these EC card readers have a PIN pad with keys or a touchscreen with displayed keys. These are typical variations for PIN code entry that are currently widespread on the market, and should therefore be understood to cover “the majority of cases” and thus not to be exhaustive.

In any case, PIN entry involves contact between the keys or touchscreen and the human hand. This is a significant drawback for the buyer, as germs, bacteria and viruses can be transmitted during contact.

Finger disinfection is required to kill them immediately. In general, however, after the PIN has been entered, there has already been further contact between the hand in question and packaging, shopping bags or even corresponding containers for the EC card when it is stored away, meaning that finger disinfection is actually too late.

The invention can reduce the transmission of germs, bacteria and viruses upon contact with the keys on the PIN pad or touchscreen. An adapter of the invention can completely or almost completely prevent the transmission of germs, bacteria and viruses upon contact with the keys of the PIN pad or touchscreen. An “almost complete prevention” of the transmission of germs, bacteria and viruses is defined by killing at least 99%, preferably at least 99.5%, most preferably at least 99.9% of the original number of germs, bacteria and viruses by means of the UV irradiation according to the invention.

As an adapter for EC card readers for the cashless purchase of goods, the payment being associated with a manual PIN entry by means of a keypad or a touchscreen, arranged in a housing of the EC card reader, in a first aspect according to the invention, the housing of the EC card reader is laterally enclosed in the region of the keypad or of the touchscreen by two opposing legs which are interconnected below the housing, and at least one of the legs projects beyond the housing on the keypad side or on the touchscreen side by means of a portion, and at least one UV light source which irradiates the whole of the keypad is arranged in the portion.

In the simplest case, the legs, together with the connection thereof extending below the housing, have a U-shape.

In an alternative embodiment to the first aspect of the invention, as an adapter for EC card readers for the cashless purchase of goods, the payment being associated with a manual PIN entry by means of a keypad or a touchscreen, arranged in a housing of the EC card reader, the housing of the EC card reader is enclosed in the region of the keypad or touchscreen by at least two opposing apparatuses (i.e., for example, on the left and right sides and/or on the upper and lower sides of the keypad or touchscreen) arranged laterally close to the keypad or touchscreen, and at least one UV light source which irradiates the whole of the keypad or touchscreen is arranged in the apparatuses. The shape, height and material properties of the apparatuses are such that the apparatuses fulfill two functions at the same time, namely (i) suitable UV irradiation of the keypad or touchscreen and (ii) a function for providing privacy from third parties such that it is extremely difficult or impossible for a third party behind or next to the user to see the PIN code being entered. Thus, the apparatuses can be configured as a pair, for example; in this case, the apparatuses are arranged only on the left and right sides of the keypad or touchscreen in spatial proximity. Furthermore, the apparatuses can be configured as a three, for example; in this case, the apparatuses are arranged on the left and right sides but also above or below the keypad or touchscreen in spatial proximity. Finally, the apparatuses can be configured as a four, for example; in this case, the apparatuses are arranged on the left and right sides as well as above and below the keypad or touchscreen in spatial proximity.

The apparatuses described above can be made, for example, of a sheet-like material that is at least 3-5 cm high and has sufficient strength.

Furthermore, in addition to the UV light source, the apparatuses described above can include a sensor, described herein, to allow the switching on and off of the UV light sources to be controlled on the basis of the presence and absence of fingers or hands.

It is more advantageous if the portion projecting beyond the housing is slightly stepped or curved or inclined towards the keypad or touchscreen without obstructing the PIN entry. The at least one UV light source in the portion of the particular leg projecting beyond the housing can thus be arranged recessed, which protects the buyer from glare during PIN entry.

Within the context of the invention, a touchscreen also includes an apparatus or display that performs the function of a keypad, but at least the function of PIN entry, preferably numeric PIN entry.

Within the scope of the invention, numeric, alphabetic or character-coded PIN entries or any mixed forms thereof are conceivable as PIN codes.

Thus, within the scope of the present invention, the terms “keypad” and “touchscreen” or similar terms can sometimes be used interchangeably in context.

In another advantageous embodiment of the adapter for EC card readers, each leg has a portion projecting beyond the housing and that preferably both are provided with at least one UV light source. This makes it easier to irradiate the whole of the keypad or touchscreen.

In one embodiment, the at least one leg, preferably both legs, can be raised and lowered automatically by a suitable mechanism and/or apparatus. For example, according to the invention, the at least one leg, preferably both legs, can thus be automatically fully or only partially raised for a cleaning or disinfection process by means of UV radiation so as to allow UV irradiation of the keypad or touchscreen in a direction from above or the side. After the cleaning or disinfection process, the at least one leg, preferably both legs, automatically moves back down to an initial position.

UV or UVC LEDs are preferably used as the UV light source.

In another advantageous embodiment, at least one leg has an element, which is resilient against the housing of the EC card reader, in order to fix the housing in place. At the same time, housings of the EC card reader with different widths can be fixed in place between the legs. Resilient elements of this kind can be curved leaf springs fastened to the inside of a leg.

It is also advantageous for there to be a free space between the connection of the legs and the bottom of the housing in order to make necessary connections to the EC card reader and/or to accommodate a battery for the at least one UV light source.

In order to reduce the irradiation of the fingers or the hand of the buyer during PIN entry, in an advantageous embodiment, at least one of the legs has a sensor which detects hand or finger movements in the scattering range of the UV light for control purposes for switching the UV light on and off. If such a movement is registered, the UV light is switched off. This further reduces radiation exposure and is also possible because disinfection has already taken place. Preferably, the UV light is turned off almost immediately or instantaneously when the aforementioned movement is registered.

If the sensor determines that there is no longer a finger or hand in the scattering range of the UV light, the UV light source is switched on again. Preferably, the UV light is turned on almost immediately or instantaneously when the aforementioned movement is registered.

A timer for the at least one UV light source can be used to switch off the UV light again after the keypad or touchscreen has not been used for a certain period of time.

The invention will be described on the basis of an exemplary embodiment. In the drawings:

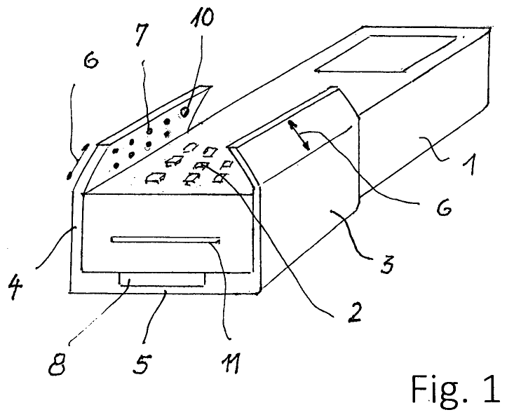

FIG. 1 is a perspective view, and

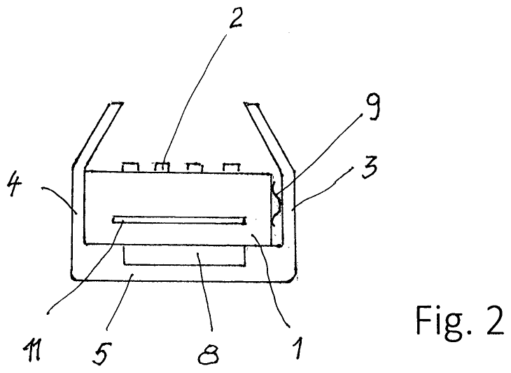

FIG. 2 is a view from the direction of the buyer.

The following description of the drawings relates to the aforementioned first aspect of the present invention:

FIG. 1 is a perspective view of an EC card reader for the cashless purchase of goods, the payment being associated with a manual PIN entry by means of a keypad 2, arranged in a housing 1 of the EC card reader, the EC card reader being fitted with the adapter according to the invention.

The adapter has two opposing legs 3, 4 which are interconnected below the housing 1 and laterally enclose the housing 1 of the EC card reader in the region of the keypad 2. The legs 3, 4 project beyond the housing 1 on the keypad side by means of a portion 6, with a UV light source 7 being arranged in the portions 6 in the form of UV LEDs which irradiate the whole of the keypad 2 when the UV LEDs are switched on. In this way, germs, bacteria and viruses are killed and can no longer be transmitted when third parties come into contact with the keypad.

The portions 6 projecting beyond the housing 1 are slightly stepped or curved or inclined towards the keypad 2. At an angle of inclination of approx. 10° to 15° towards the keypad 2, the operability thereof is not restricted, but the light sources 7 can be arranged recessed for radiation protection, without impairing the irradiation of the keypad 2.

A sensor 10 is arranged in the portion 6 of the leg 4, the sensor switching the UV light on and off on the basis of hand or finger movements in the scattering range of the UV light. This also minimizes radiation exposure.

The sensor 10 can also be coupled to a timer for the UV light sources 7 such that the UV light is switched off after the keypad 2 has not been used for a certain period of time.

It is further shown that, between the connection 5 of the legs 3, 4 and the bottom of the housing 1, there is a free space 8 for establishing necessary connections to the EC card reader and/or for accommodating a battery for the UV light sources 7.

The outer surfaces of the legs 3, 4 can be used for advertising purposes.

FIG. 2 shows a view from the buyer's perspective, together with the insertion slot 11 for an EC card.

In addition, it is shown that, on the inside wall, the leg 3 has an element 9, which is resilient against the housing 1 of the EC card reader, in order to fix the housing 1 in place and to compensate for different housing widths.

In this case, a curved leaf spring is used as the resilient element 9. In the following, reference can be made to the description of FIG. 1.

### LIST OF REFERENCE SIGNS

1 Housing of EC card reader
2 Keypad
3 Leg
4 Leg
5 Connection of the legs
6 Portion projecting beyond the housing
7 UV light source
8 Free space below the housing
9 Resilient element
10 Sensor
11 Insertion slot for EC card

The above-described drawings describe an exemplary embodiment according to the invention for a keypad arranged on the EC card reader. It is clear to a person skilled in the art that this can also be transferred in an analogous or suitably similar manner to an exemplary embodiment with a touchscreen arranged on the EC card reader.

Together with the above disclosure, the invention further includes the following embodiments:

First, an adapter for EC card readers for the cashless purchase of goods, the payment being associated with a manual PIN entry by means of a keypad (2) or a touchscreen, arranged in a housing (1) of the EC card reader, characterized in that the housing (1) of the EC card reader is laterally enclosed in the region of the keypad (2) or of the touchscreen by two opposing legs (3, 4) which are interconnected below the housing (1), at least one of the legs (3, 4) projects beyond the housing (1) on the keypad side or on the touchscreen side by means of a portion (6), and at least one UV light source (7) which irradiates the whole of the keypad (2) or touchscreen is arranged in the portion (6).

Second, the adapter according to the first Embodiment, characterized in that the legs (3, 4), together with the connection (5) thereof, have a U-shape.

Third, the adapter according to the first of second Embodiment, characterized in that the portion (6) projecting beyond the housing (1) is slightly stepped or curved or inclined towards the keypad (2) or touchscreen.

Fourth the adapter according to any one of the first to third Embodiments, characterized in that each leg (3, 4) has a portion (6) projecting beyond the housing (1) and preferably both are provided with at least one UV light source (7).

Fifth the adapter according to any one of the first to fourth Embodiments, characterized in that the UV light source (7) is UV or UVC LEDs.

Sixth the adapter according to any one of the first to fifth Embodiments, characterized in that the at least one UV light source (7) is arranged recessed in the particular leg (3, 4).

Seventh, the adapter according to any one of the first to sixth Embodiments, characterized in that at least one leg (3 or 4) has an element (9), which is resilient against the housing (1) of the EC card reader, in order to fix the housing 1 in place.

Eighth, the adapter according to any one of the first to seventh Embodiments, characterized in that, between the connection (5) of the legs (3, 4) and the bottom of the housing (1), there is a free space (8) for establishing necessary connections to the EC card reader and/or for accommodating a battery for the at least one UV light source (7).

Ninth, the adapter according to any one of the first to eighth Embodiments, characterized in that at least one of the legs (3 or 4) has a sensor (10) which detects hand or finger movements in the scattering range of the UV light for control purposes for switching the UV light on and off.

Tenth, the adapter according to any one of the first to ninth Embodiments, characterized in that a timer is provided for the at least one UV light source (7), the timer switching off the UV light after the keypad (2) or touchscreen has not been used for a certain period of time.

A second aspect of the invention is directed to the following: The invention further relates to an adapter and an apparatus for disinfecting devices to be operated by the human hand, in particular card readers, which, in the form of a mobile or stationary terminal, are used for the cashless purchase of goods using the electronic cash method, in which a PIN code must be entered upon payment. To enter the PIN code, these EC card readers have a PIN pad with keys or a touchscreen with displayed keys.

Furthermore, the adapter is also configured in such a way as to be able to be mounted on terminals in which the cards are additionally pulled through a scanning slot or can be read by holding them up to a scanner at the side.

In principle, the adapter according to the first and second aspects of the invention is configured so as to also be able to be used for “contactless payments.” Within the context of the invention, “contactless” means that it is not necessarily the case that a PIN code is entered and direct contact is established between a hand and/or finger and a keypad and/or touchscreen; rather, a cash card such as an EC card, a Visa card, a Mastercard, etc. is simply tapped. In this case, the UV radiation according to the invention can also disinfect or clean the contact surface for the cash card or, e.g., a smartphone (see below) in such a way that germs, bacteria and viruses cannot be transmitted to the user via the card itself, or in a way that does not transmit diseases.

The invention is in principle not limited to EC cards but extends in principle to cash card payments (Visa, Diner's Club, Mastercard etc.) but also to other payment methods such as payment via smartphone on a read-out apparatus.

In any case, PIN entry involves contact between the keys and the human hand and/or fingers. This is a significant drawback for the buyer, as germs, bacteria and viruses can be transmitted during contact.

The same applies to all devices that can be operated with the hand or fingers.

The same applies to the above-mentioned details regarding “contactless payments,” i.e., simply tapping or bringing close a cash card or smartphone with a readout apparatus, e.g., a technical contact surface arranged on a payment apparatus.

Finger disinfection is required to immediately kill these germs, bacteria and viruses on card readers.

In general, however, after the PIN has been entered, there has already been further contact between the hand in question and packaging, shopping bags or even corresponding containers for the EC card when it is stored away, meaning that finger disinfection is actually too late.

Alternatively, it would also be possible to wipe the keys or the touchscreen with a disinfectant, but this would be time-consuming for the sales staff and also expensive in the long run.

Another object of the invention is as follows:

The invention can reduce, in a simpler way, the transmission of germs, bacteria and viruses upon contact with devices and, in particular, with the keys of the PIN pad or touchscreen.

As an adapter for a card reader for the cashless purchase of goods, wherein a manual PIN entry by means of a keypad or a touchscreen, arranged in a housing of the card reader, is also provided for the payment, in a second aspect according to the invention, that the adapter consists of an adapter housing having least one lateral leg, the lateral leg being connectable or connected to a side of the housing of the card reader or to a receptacle for the card reader, the adapter housing having a portion which is arranged above the keypad or touchscreen and on which or on the inside of which at least one UV light source is located, the UV light therefrom irradiating the keypad or touchscreen from above.

Within the context of the invention, in some embodiments, “irradiated from above” does not necessarily mean that the UV rays radiate at a 90° angle or at a substantially 90° angle to the surface of the keypad or touchscreen. Rather, “irradiated from above” can also include lateral irradiation in some embodiments of the invention, i.e., with an angle of incidence of less than 90° in relation to the surface of the keypad or touchscreen as the base plane.

In one embodiment of the invention, the UV light thus irradiates the keypad or touchscreen at a suitable angle of the UV rays such that the UV rays do not radiate perpendicularly but at an angle of <90°, preferably <80°, more preferably <70°, even more preferably <45° with respect to the plane of the keypad or touchscreen.

Preferably, the adapter housing has two lateral legs which are connected by the portion and are each connectable or connected to a side of the housing of the card reader or to a receptacle for the card reader.

In an advantageous configuration, the portion of the adapter housing above the keypad or above the touchscreen is curved and the inner curved face of the portion faces towards the keypad or touchscreen. The inner curved face is preferably semi-circular or semi-elliptical, similar to an archway. Within the context of the invention, this alternative can be referred to as a “UV curve” as an apparatus.

In one embodiment of the second aspect of the invention, the “UV curve” can be raised and lowered automatically by a suitable mechanism and/or apparatus. For example, according to the invention, the UV curve can thus be automatically fully or only partially raised for a cleaning or disinfection process by means of UV radiation so as to allow UV irradiation of the keypad or touchscreen in a direction from above or the side. After the cleaning or disinfection process, the UV curve automatically moves back down to an initial position.

The term “keypad” includes a touchscreen and also a fingerprint scanner.

Preferably, multiple UV light sources are arranged and distributed over the entirety of the inside of the portion.

Irradiating the keypad from above or from the side has multiple advantages. There is no collision with an arranged privacy screen for the PIN entry and with a scanning slot or side scanning surface.

Furthermore, in the case of a curved portion, the UV radiation can be directed more precisely at the keypad or touchscreen because the inner curve acts like a magnifying glass such that the UV rays are focused towards the keypad or touchscreen.

It is further provided that the portion is convex on the inside in cross section. This allows the width (b) of the portion to be kept narrower than the length (1) of the keypad or touchscreen.

The convex configuration allows the UV rays to be distributed in relation to the keypad length or touchscreen length. Thus, all regions of the keypad or touchscreen are optimally irradiated with UV light, without having to match the width (b) of the curved portion to the particular length of the keypad or touchscreen.

Furthermore, in an advantageous embodiment of the adapter, the lateral leg or legs, after the region of the connection thereof to the housing of the card reader or to the receptacle of the card reader, are angled in the longitudinal extensions thereof so as to form an obtuse angle (a). Firstly, this has the advantage that the width (b) of the curved portion appears narrower to the user when the PIN is entered, and secondly, the user's view of the keypad and the viewing window with the indication of the payment amount is optimal.

Spacers are preferably provided as connecting means between the two legs of the adapter housing and the housing of the card reader or the receptacle of the card reader, the spacers being insertable or screwable by means of the shaft thereof into the legs and preferably having, on the other side of the shaft, an angled part which encloses an edge of the housing of the card reader or the receptacle of the card reader.

The shaft of the spacer is preferably length-adjustable or is provided in different lengths adapted to different housings of the card reader or to different receptacles of the card reader.

In a further advantageous embodiment, the shaft can be fixed in lateral legs in different positions in relation to the axis of rotation of the shaft. In this way, fine adjustment of the adapter in relation to the keypad or touchscreen can be achieved.

In further advantageous embodiments:
the UV light source is UV or UVC LEDs,
the UV light sources are recessed in the portion to eliminate possible glare effects,
the adapter housing has a sensor which detects hand or finger movements in the scattering range of the UV light for control purposes for switching the UV light on and off such that the fingers or the hands are not unnecessarily irradiated, and/or
a timer for the UV light sources is provided, which switches off the UV light after the keypad has not been used for a certain period of time.

A third aspect of the invention is directed to the following:

Another part of the invention is an apparatus for disinfecting devices to be operated by the human hand, wherein a three-dimensional structure is provided, which, in the X dimension, has a curved surface directed inwardly into the structure, or the structure itself has a curved shape, and in either case, the curved inner surface is directed the particular device and the curved inner surface is provided, in at least one portion, with UV light sources which directly irradiate the device with UV light.

The curved configuration of the apparatus allows the UV radiation to be focused more precisely at the device because the curve acts like a magnifying glass such that the UV rays are focused towards the device to be irradiated. In this way, the disinfection effect is increased even when the light intensity decreases.

In an advantageous embodiment, the curved inner surface has a convex shape in the Z dimension in relation to the cross section. This results in an extension of the irradiated area in the X direction. The structure can thus be kept narrower in the X dimension than the area to be irradiated.

X and Z denote dimensions in the Cartesian coordinate system, by way of example.

Depending on the device, the irradiation can be from above, from below or even from the side. The attachment must be done accordingly. This can be an adapter solution as described before, or the structure is arranged on stands, etc. or on objects on which the devices to be disinfected are also arranged.

The invention will be described with reference to the drawings for another adapter of a card reader. In the drawings:

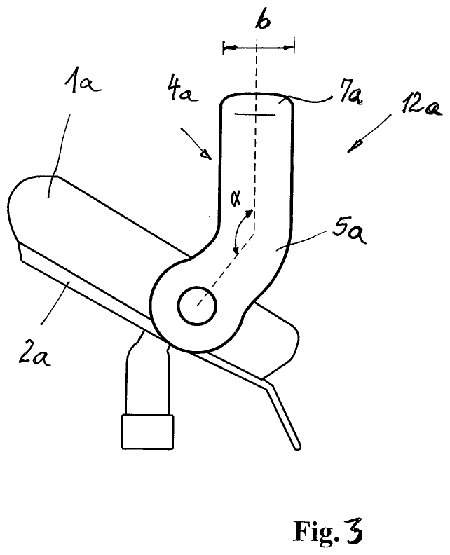

FIG. 3 is a side view of an adapter on a card reader,

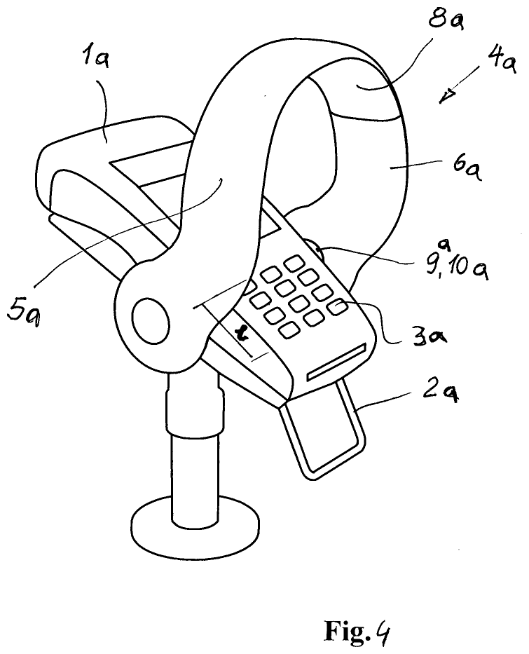

FIG. 4 is a perspective view of the adapter on a card reader, and

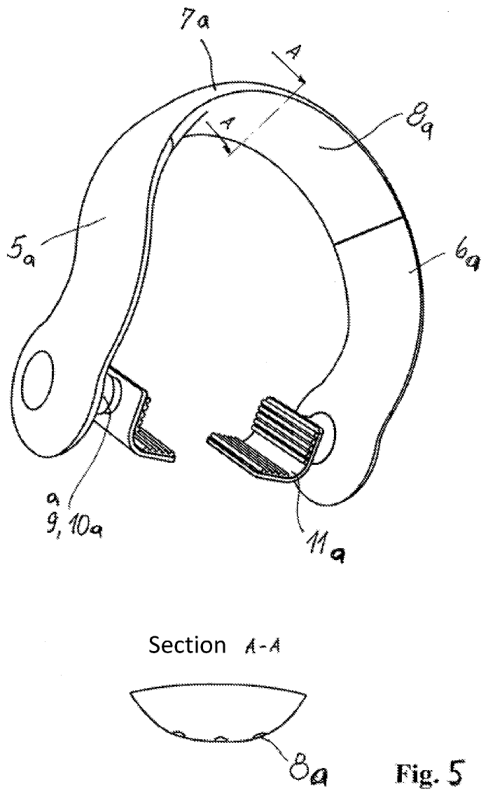

FIG. 5 is a perspective view of the adapter.

FIG. 3 is a side view of a card reader arranged in a receptacle 2a and having a housing 1a. The adapter is arranged on the housing 1a of the card reader by means of the legs 5a, 6a which are connected by a curved portion 7a having the width b. In order to ensure optimal viewing conditions (viewing direction 12a) for the user entering the PIN, the lateral legs 5a, 6a, after the region of the connection thereof to the housing 1a, are angled in the longitudinal extensions thereof so as to form an obtuse angle a. Preferably, the angle a is between 130° and 160°.

In addition, the viewing direction 12a is currently directed at the width b of the curved portion 7a of the adapter housing 4a at an angle and makes the width appear shorter.

FIG. 4 is a perspective view of the above-described card reader together with an adapter. The adapter housing 4a is connected at the lateral legs 5a, 6a to the housing 1a of the card reader via spacers 9a. The spacers 9a allow the adapter housing 4a to pivot to a limited extent relative to the housing 1a of the card reader to allow fine adjustments to be made on site. The connection should subsequently be fixed. It would also be possible to provide a stepped grid for the pivoting.

The UV light sources 8a, in the form of rows of LEDs in this case, are arranged in or on the curved portion 7a on the inner face. The UV light sources irradiate the keypad 3a from above, with the curved arrangement allowing the UV rays to be focused on the keypad 3a.

Furthermore, the curved portion has a convex shape on the inner surface in the cross section of the width b. This results in complete coverage of the keypad 3a with UV radiation, even though the width b of the adapter housing 4a is smaller than the length 1 of the keypad 3a.

FIG. 5 shows the adapter alone. In a section A-A through the curved portion, the convex shape thereof can be seen.

The spacers 9a are used to attach the adapter housing 4a to the housing 1a of the card reader or to the receptacle 2a thereof. The spacers have a shaft 10a by means of which the spacers can be inserted or screwed into an opening in the particular leg 5a, 6a. On the other side of the shaft 10a, there is an angled part 11a which grips around an edge portion of the housing 1a of the card reader or the receptacle 2a thereof when the connection to the housing or receptacle is established. The shaft 10a of the spacers 9a is length-adjustable in order to mount the adapter housing 4a on different housings 1a of the card readers or different receptacles 2a. Alternatively, it would also be possible to offer different shaft lengths.

In this way, it is possible not only to fix adapter housings 4a to different card readers or to the receptacles thereof, but also to provide the necessary space for an additional scanning slot or side surface scanning. The adapter with its adapter housing is not changed.

### LIST OF REFERENCE SIGNS

1a Housing of the card reader
2a Receptacle for the card reader
3a Keypad
4a Adapter housing
5a Lateral leg of the adapter housing
6a Lateral leg of the adapter housing
7a Portion of the adapter housing
8a UV light source
9a Spacers
10a Shaft of the spacer
11a Angled part of the spacer
12a Viewing direction of the user during PIN entry
1 Length of the keypad
b Width of the curved portion

Within the above-mentioned context of the invention in relation to the second and third aspects, the invention further comprises the following numbered embodiments:

Eleventh—An adapter for disinfecting a keypad (3) or a touchscreen of a card reader for cashless payment, wherein a manual PIN entry by means of the keypad (3) or a touchscreen, arranged in a housing (1) of the card reader, is also provided for the payment, characterized in that the adapter consists of an adapter housing (4) having least one lateral leg (5), the lateral leg (5) being connectable or connected to a side of the housing (1) of the card reader or to a receptacle (2) for the card reader, the adapter housing having a portion (7) which is arranged above the keypad (3) or touchscreen and on which or on the inside of which at least one UV light source (8) is located, the UV light therefrom irradiating the keypad (3) or touchscreen from above.

Twelfth—The adapter according to the eleventh Embodiment, characterized in that the adapter housing (4) has two lateral legs (5, 6), which are connected by the portion (7) and are each connectable or connected to a side of the housing (1) of the card reader or to a receptacle (2) for the card reader.

Thirteenth—An adapter according to the eleventh or twelfth Embodiment, characterized in that the portion (7) is curved and the inner curved face thereof faces towards the keypad (3) or touchscreen.

Fourteenth—The adapter according to any of the eleventh to thirteenth Embodiments wherein the UV light sources (8) are distributed over the entirety of the inside of the portion (7).

Fifteenth—The adapter according to the eleventh or twelfth Embodiments, characterized in that the portion (7) is convex on the inside in cross section.

Sixteenth—The adapter according to any one of the eleventh to fifteenth characterized in that the at least one lateral leg (5) or both legs (5, 6), after the region of the connection thereof to the housing (1) of the card reader or to the receptacle (2) of the card reader, are angled in the longitudinal extensions thereof so as to form an obtuse angle (a).

Seventeenth—The adapter according to any one of the twelfth to sixteenth Embodiments, characterized in that spacers (9) are used as connecting means between the legs (5, 6) and the housing (1) of the card reader or the receptacle (2) of the card reader, the spacers being insertable or screwable by means of the shaft (10) thereof into the legs (5, 6) and having, on the other side of the shaft (10), an angled part (11) which encloses an edge of the housing (1) of the card reader or the receptacle (2) of the card reader.

Eighteenth—The adapter according to any one of the twelfth to seventeenth Embodiments, characterized in that the shaft (10) of the spacer (9) is length-adjustable or is provided in different lengths adapted to the housing (1) of the card reader or the receptacle (2) of the card reader.

Nineteenth—The adapter according to the seventeenth or eighteenth Embodiments, characterized in that the shaft (10) is able to be fixed in the lateral parts (5, 6) in different positions in relation to the axis of rotation of the shaft (10).

Twentieth—The adapter according to any one of the eleventh to nineteenth Embodiments, characterized in that the UV light source (8) is UV or UVC LEDs.

Twenty-first—The adapter according to any one of the eleventh to twentieth Embodiments, characterized in that the UV light sources (8) are arranged recessed in the portion (7).

Twenty-second—The adapter according to any one of the eleventh to twenty-first Embodiments, characterized in that the adapter housing (4) has a sensor (10) which detects hand or finger movements in the scattering range of the UV light for control purposes for switching the UV light on and off

Twenty-third—The adapter according to any one of the eleventh to twenty-second Embodiments, characterized in that a timer for the UV light sources (8) is provided, which switches off the UV light after the keypad (3) or touchscreen has not been used for a certain period of time.

Twenty-fourth—An apparatus for disinfecting devices to be operated by the human hand, characterized in that a three-dimensional structure is provided, which, in the X dimension, has a curved surface directed inwardly into the structure, or the structure itself has a curved shape, and in either case, the curved inner surface is directed at the particular device and the curved inner surface is provided, in at least one portion, with UV light sources which directly irradiate the device with UV light.

Twenty-fifth—An apparatus according to the twenty-fourth Embodiment, characterized in that the curved inner surface has a convex shape in relation to the Z dimension.

1. An adapter for an EC card reader including a housing and a keypad or a touchscreen for manual PIN (personal identification number) entry, the adapter comprising:
at least two opposing legs interconnected below the housing, at least a portion of at least one of the legs projecting beyond the housing on a side of the keypad or the touchscreen, and at least one UV light source arranged in the at least one portion to irradiate an entirety of the keypad or touchscreen.

2. The adapter according to claim 1, wherein the at legs and a connection thereof define a U-shape.

3. The adapter according to claim 1, wherein the at least one portion is stepped, curved or inclined towards the keypad or touchscreen.

4. The adapter according to claim 1, wherein each of the legs comprises a portion projecting beyond the housing and at least one UV light source.

5. The adapter according to claim 1, wherein the UV light source comprises UV or UVC LEDs.

6. The adapter according to claim 1, wherein the at least one UV light source is recessed in the at least one portion.

7. The adapter according to claim 1, comprising a spacer resilient against the housing configured to fix the housing.

8. The adapter according to claim 1, comprising a free space between a connection of the legs and a bottom of the housing, the free space being configured to permit connections to the EC card reader and/or to accommodate a battery for the at least one UV light source.

9. The adapter according to claim 1, movement sensor configured to detect hand or finger movements in the scattering range of the UV light and to switch the UV light on and off.

10. The adapter according to claim 1, comprising a timer configured to switch off the at least one UV light source after the keypad or touchscreen has not been used for a certain period of time.

11. The adapter according to claim 1, wherein the at least one portion projects by a height of at least 3-5 cm, to provide privacy from third parties during PIN code entry.

12. The adapter according to claim 1, wherein the housing is detachably mounted on the EC card reader.

13. An adapter for disinfecting a keypad or a touchscreen of a card reader for cashless payment, the card ready allowing a manual PIN entry by means of the keypad or touchscreen, comprising:
an adapter housing having at least one lateral leg, the lateral leg being configured to connect to a side of a housing of the card reader or to a receptacle for the card reader, said adapter housing having a portion arranged above the keypad or touchscreen, the portion comprising at least one UV light source being positioned to irradiate the keypad or touchscreen from above.

14. The adapter according to claim 13, wherein the adapter housing comprising two lateral legs connected by the portion and configured to connect to a side of the housing of the card reader or to a receptacle for the card reader.

15. The adapter according to claim 13, wherein the portion is curved and comprising an inner curve face configured to face toward the keypad or touchscreen.

16. The adapter according to claim 13, comprising a plurality of UV light sources distributed over an entirety of the inside of the portion.

17. The adapter according to claim 13, wherein the portion is convex in cross-section.

18. The adapter according to claim 13, wherein the at least one lateral leg is angled in the longitudinal extensions thereof to form an obtuse angle.

19. The adapter according to claim 13, comprising spacers between the legs and the housing, the spacers being insertable or screwable into the legs comprising an angled part configured to enclose an edge of the housing of the card reader or the receptacle of the card reader.

20. The adapter according to claim 19, wherein the spacer is length adjustable.

21. The adapter according to claim 19, wherein the spacer comprises a shaft that can be fixed in the legs in different positions in relation to the axis of rotation of the shaft.

22. The adapter according to claim 13, wherein the UV light source comprises UV or UVC LEDs.

23. The adapter according to claim 13, wherein UV light source is recessed in the portion.

24. The adapter according to claim 13, comprising a movement sensor configured to detect hand or finger movements in a scattering range of the UV light source, the movement sensor being configured to control switching the UV light source on and off.

25. The adapter according to claim 13, comprising a timer configured to switch off the UV light after the keypad or touchscreen has not been used for a certain period of time.

26. The adapter according to claim 13, configured to automatically move between a folded-down initial position and a position suitable for disinfection or cleaning.

27. An apparatus for disinfecting a device to be operated by the human hand, comprising a three-dimensional structure, which, in the X dimension, has a curved surface directed inwardly into the structure, or the structure itself has a curved shape, the curved inner surface or the curved shaped comprising UV light sources configured to directly irradiate the device with UV light.

28. The apparatus according to claim 27, wherein the curved inner surface or the curved shape has a convex shape in relation to the Z dimension.

29. The adapter according to claim 27, wherein the UV light sources are arranged at a suitable inclination to irradiate the device of the range of 90° to 10° with respect to the keypad or touchscreen of the device.
